(19) United States
(12) Patent Application Publication
(10) Pub. No.: US 2010/0307499 A1
(43) Pub. Date: Dec. 9, 2010

(54) RESPIRATOR WITH AUTOMATICALLY CONTROLLED PRESSURE-ASSIST RESPIRATION

(75) Inventors: Marcus EGER, Lubeck (DE); Thomas HANDZSUJ, Lubeck (DE); Zhanqi ZHAO, Freiburg (DE); Knut MÖLLER, Kirchzarten (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 12/754,055

(22) Filed: Apr. 5, 2010

(30) Foreign Application Priority Data
Jun. 5, 2009 (DE) 10 2009 023 965.0

(51) Int. Cl. A61M 16/00

(52) U.S. Cl. 128/204.23

(57) ABSTRACT

A respirator with an adjustable pressure or volume flow curve has a control and analyzing unit, which is set up to determine the resistance R and the alveolar pressure Pₐₗᵥ(t). The control and analyzing unit checks the functional dependence of Pₐₗᵥ(t) and of the tidal volume Vol(t) for time intervals in which an indicator of the quality of a linear functional dependence of Pₐₗᵥ(t) and Vol(t) meets a preset threshold criterion and to determine the elastance E or compliance C from the rise of the alveolar pressure Pₐₗᵥ(t) as a function of the volume Vol(t) only in the time intervals thus determined.

### CROSS REFERENCE TO RELATED APPLICATIONS

This application claims the benefit of priority under 35 U.S.C. §119 of German Patent Application DE 10 2009 023 965.0 filed Jun. 5, 2009, the entire contents of which are incorporated herein by reference.

### FIELD OF THE INVENTION

The present invention pertains to a respirator (also known as a ventilator) and to a process for automatically controlling a respirator for pressure-assist ventilation, wherein the respirator has a fan for feeding breathing gas with an adjustable pressure, means for recording measured values for the airway pressure P_{aw}(t) and volume flow flow(t) and for determining the tidal volume Vol(t) and with a control and analyzing unit for controlling the respirator for pressure-assist ventilation with the use of the mechanical parameters of the lungs, namely, resistance and elastance or compliance, which are determined automatically according to the process and the respirator.

### BACKGROUND OF THE INVENTION

The goal of respiration is to assist the respiratory muscles in order to achieve sufficient oxygen supply (oxygenation) and carbon dioxide removal. If the mechanical properties of the lungs (resistance and elastance (=1/compliance)) of a patient are known, the spontaneous respiratory activity can be determined with these parameters by calculation. The respirator can then fully perform the respiratory work regardless of the patient's breathing activity or, in case of assisted process, facilitate the respiratory work. The patient is passive in the first case, for example, due to sedation, and must rely on complete respiration. The patient is breathing spontaneously in the second case and must only be assisted by the respirator. The difficult task of establishing synchronicity between the patient and the respirator arises here. Spontaneously breathing patients were often sedated in the past in order to set the respiration correctly and to force the synchronicity between the patient and the respirator. This procedure is no longer acceptable at the current state of knowledge because intubation may be necessary due to the risk of obstruction of the airways and there is a great risk of damage to the lungs due to active breathing. Maintaining spontaneous breathing is nowadays a highly desirable goal in clinical treatment.

Knowledge of the mechanical parameters of the lungs is necessary to set the assist parameters Flow Assist (FA) and Volume Assist (VA) in case of proportional assist ventilation (PAV or PPS). Adjustment of assist is necessary not only at the beginning, but also time and time again since the mechanical properties of the lungs may change in case of repositioning or at the time of onset of obstruction with mucus. Since this can be achieved in clinical routine only with difficulty and physicians are justifiably afraid of causing run-aways (i.e., overcompensation, which leads to instability of the mode of respiration and could put the patient at risk if the alarm limits are set incorrectly or at least increases the patient's respiratory work), this type of proportional assist ventilation has met with limited acceptance only in practice despite its physiological advantages.

Pressure assist, which contains a component proportional to the volume flow (flow) currently present as well as a component proportional to the volume, is generated in “Proportional Assist Ventilation” methods (cf., e.g., Younes, M.: “Proportional Assist Ventilation,” in: Tobin M. J., ed. “Principles and practice of mechanical ventilation,” New York, McGraw-Hill, 1994, pages 349-369). The degree of assist is predetermined by the set values flow assist (FA) and volume assist (VA). Due to the positive feedback of the volume flow and of volume, this form of respiration leads to a kind of servo control, which makes it possible to compensate separate components of the resistive and elastic resistances of the respiratory system and hence to quantitatively take over respiratory work from the patient. However, a sufficiently accurate estimated value must be available for this for the actual resistance (R) and elastance (E), because instabilities (the so-called run-aways) and possibly damage to the lungs due to barotraumas may otherwise occur.

Furthermore, efforts have been made for a rather long time now to reliably determine R and E during spontaneous breathing in a minimally invasive manner (cf., e.g., WO 97/22377 A1). The special difficulties lie in the fact that the patient's spontaneous breathing activity may cause highly incorrect estimates in the determination of the mechanical parameters of breathing. A known procedure is the introduction of disturbing maneuvers in the breathing pattern (e.g., by a brief occlusion) at points in time at which a passive breathing pause is assumed, and the subsequent analysis of the disturbed respiratory signals. However, it is not guaranteed that the patient is in an undisturbed phase of the breathing cycle at the time of the maneuver, and therefore the validity of the measurement is not guaranteed; it also cannot be demonstrated later. This is due to the fact that the activity of the respiratory muscles cannot be isolated from the respirator-controlled breathing pattern because of close correlations either on the basis of signal theory or statistics.

The respirator PB840 with the PAV+ respiration mode, which is said to provide for an automatic setting of assist, is available commercially from the companies Covidien/Tyco/Puritan/Bennett. However, the parameters determined for the resistance (R) and elastance (E) or compliance (C=1/E) are inaccurate, so that reliable compensation of the respiratory work is possible at low degrees of assist (i.e., at low assist parameters FA and VA) only.

Respirator PB840 with the implemented PAV+ method also uses occlusions, i.e., brief closures of the breathing gas feed line to the patient, always after the end of inspiration by the patient. These occlusions are relatively long (300 msec) and therefore they markedly interfere with the patient's breathing pattern. Furthermore, the occlusion takes place at a point in time of the breathing cycle during which there often is intense respiratory activity on the part of the patient, contrary to what should be presumed for the effectiveness of the method. This leads to errors in the calculation of the mechanical parameters of the lungs, namely, resistance (R) and elastance (E) as well as to a wide spread of the numerical values. Since these parameters R and E are used to set the assist parameters FA and VA, reliable compensation of the respiratory work is consequently possible within the framework of low assist parameters only.

### SUMMARY OF THE INVENTION

The object of the present invention is to provide a respirator and a process for automatically controlling same, by means of which the elastance or compliance can be determined more accurately and more reliably.

The respirator according to the present invention has a fan for feeding breathing gas with an adjustable pressure or volume flow curve; means for recording measured values for the airway pressure P_{aw}(t) and volume flow Flow(t) and for determining the tidal volume Vol(t), and a control and analyzing unit. The control and analyzing unit is set up at first to determine the resistance R, for which the method described in patent application EP 1972274 A1 (see also US 2008234595 (A1) which is hereby incorporated by reference) with the use of very brief occlusions of about 100 msec can be used; as an alternative, the resistance can be determined by the method described in the article “Proportional Assist Ventilation” by Younes, which was discussed above, or by a conventional method, in which the patient is passive (for example, sedated), or by any other prior-art method. The first-named method for determining the resistance (compliance) by means of repeated brief occlusions (so-called P0.1 occlusions) is preferred because these P0.1 occlusions are so short that they are hardly perceived by the patient and therefore do not interfere with the breathing activity. Furthermore, they are clinically accepted and are used repeatedly, for example, within the framework of weaning to measure the respiratory drive.

The control and analyzing unit is set up to determine the resistive pressure component Flow(t)*R from the measured value for the volume flow Flow(t) and the value determined for resistance R, and to subtract this component from the measured airway pressure P_{aw}(t) in order to thus determine the alveolar pressure Pₐₗᵥ(t) and to plot it as a function of time.

The control and analyzing unit is set up, furthermore, according to the present invention to analyze the functional dependence of Pₐₗᵥ(t) and Vol(t) and to search it for certain time intervals, namely, by checking the dependence of Pₐₗᵥ(t) and Vol(t) with variable time interval lengths for time intervals during which an indicator for the quality of a linear functional dependence of Pₐₗᵥ(t) and Vol(t) meets a preset threshold criterion, i.e., a search is performed with variable interval lengths for time intervals during which there are only minimal deviations from linearity, so that phases, during which there is a highly variable spontaneous breathing, are omitted in this manner, so that calculation errors based on spontaneous breathing are reduced. The elastance or compliance is finally determined only in the time intervals thus determined from the rise of the alveolar pressure Pₐₗᵥ(t) as a function of the volume Vol(t).

As an indicator of the quality of the linear functional dependence, linear regressions can be performed in the time intervals with variable interval length and a function cumulating the residues of the linear regression, e.g., the variance, can be subjected to a predetermined threshold criterion, the variance being obtained from the sum of the square residues (deviations of the straight lines from the measured points). The variance can be used, for example, to determine the confidence limits to a preset percentage, preferably the 95% confidence limit, for the parameter determined by the linear regression, and these confidence limits can be subjected to a predetermined threshold criterion.

It was found that an especially sensitive indicator is obtained for the quality of the linear functional dependence by determining the difference of the confidence limits of the elastance determined in the regression and standardizing this difference for the root of the number of the data values in the time interval and the value determined in the regression for the elastance; this value, formed in this manner, quasi represents a standardized error interval, which can be required to be below a preset threshold value, which means that the elastance (compliance) determined in the linear regression has a small error.

As an alternative, the control and analyzing unit may be set up to perform linear regressions in the time intervals with variable interval lengths as an indicator of the quality of the linear functional dependence and to use the correlation coefficient of the respective regressions as an indicator of the quality, a minimal deviation of the correlation coefficient from 1 being allowed as a preset threshold criterion.

The control and analyzing unit is preferably set up, furthermore, if a plurality of time intervals that meet the threshold criterion are determined within a breathing cycle, to sum up the values calculated for the elastance (or compliance) during these time intervals by performing first a freak value test and discarding detected freak values and summing up the remaining values into a mean or median value. In addition, the control and analyzing unit may be set up to sum up the values calculated for the elastance (or compliance) in a plurality of time intervals over consecutive breaths by performing first a freak value test and discarding detected freak values and summing up the remaining values into a mean or median value.

The elastance or compliance determined according to the present invention with the device or the process can then be used together with the resistance determined in another manner for various purposes as follows:
to set the degree of assist in case of proportional pressure support (Proportional Pressure Support or Proportional Assist Ventilation);
to automatically set a ramp in Pressure Support as described in DE 10 2007 033 546 B3;
for the use of gas exchange models for optimized respiration;
for diagnostic or monitoring purposes;
to calculate the respiratory muscle pressure (Pₘᵤₛ);
to trigger or terminate breathing strokes therewith;
to be used as a difference signal for scaling a non-pneumatic muscle activity signal, for example, the sEMG signal, as described in patent application DE 10 2007 062 214;
to be used in the determination of the degree of respiratory muscle exhaustion;
to be used in a strategy for weaning the patient from respiration;
to be indicated generally for diagnostic or monitoring purposes;
to be used for supporting medical decisions, for example, for the early determination of the point in time of extubation.

The present invention will be explained in more detail below on the basis of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a graphical representation showing a dependence of the volume on the alveolar pressure for an exemplary breath;

FIG. 2 is a graphical representation showing a dependence of the volume, of the alveolar pressure and of the estimated muscle pressure on the time for the same exemplary breath from FIG. 1; and

FIG. 3 is a schematic diagram showing features of a respirator according to the invention.

### DESCRIPTION OF THE PREFERRED EMBODIMENTS

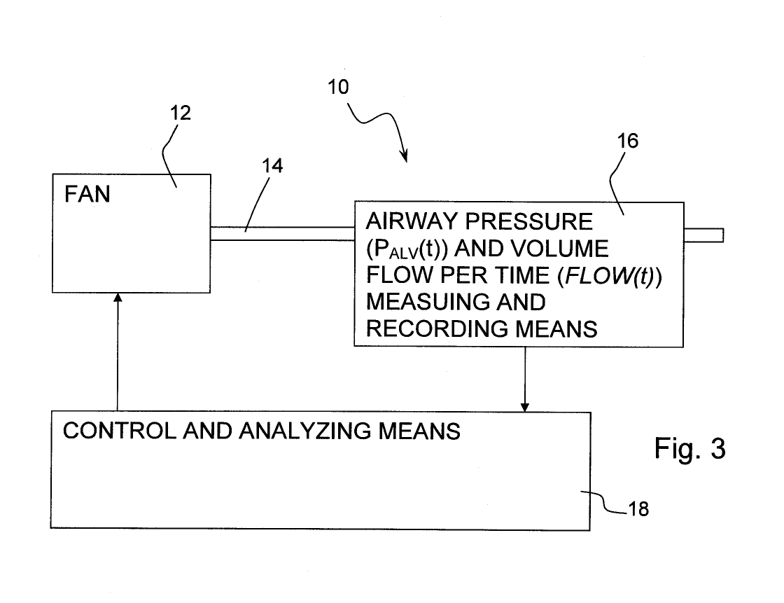

Referring to the drawings in particular, a respirator generally designated 10 is provided with a fan 12 for feeding breathing gas with an adjustable pressure or volume flow curve via a breathing line 14 (FIG. 3). Values for the airway pressure (P_{aw}(t)) and volume flow per time (Flow(t)) are provided with a recording means 16. The recording means 16 records the measured values for the airway pressure (P_{aw}(t)) and volume flow per time (Flow(t)) for a control and analyzing unit or control and analyzing means 18. The elastance and compliance can be readily determined with the control means during phases during which the respiratory muscle activity remains sufficiently constant. Assuming a one-compartment model, the so-called motion equation applies to the relationship between muscle activity Pₘᵤₛ(t), the mechanical parameters of the lungs (R and E) and the respiration signals P_{aw}(t), volume flow Flow(t), and volume Vol(t):

Pₘᵤₛ(t)+P_{aw}(t)=R*Flow(t)+E*Vol(t)+PEEPi

where PEEPi is the so-called intrinsic PEEP (positive end-expiratory pressure), i.e., the (relatively constant) pressure remaining in the lungs after expiration. When assuming that the muscle activity is constant during a time window, i.e., Pₘᵤₛ(t)=K, and subtracting the resistive pressure component R*Flow(t) from the airway pressure P_{aw}(t), the alveolar pressure Pₐₗᵥ(t) is obtained as

Pₐₗᵥ(t)=E*Vol(t)+PEEPi−K.

This equation shows that the elastance E can be determined by means of regression between the variables Pₐₗᵥ(t) and Vol(t) assuming a constant muscle activity Pₘᵤₛ(t)=K.

However, Pₘᵤₛ(t) is changing continually in spontaneously breathing patients. An essential feature of the present invention is that time intervals or time windows are automatically found in which Pₘᵤₛ(t) is sufficiently constant, and these time intervals are identified by time intervals with variable interval length being shifted one after another over the breath cycle and a linear regression of Pₐₗᵥ(t) and Vol(t) being performed and an indicator for the quality of the adaptation being determined. The variance of the particular linear regression or the 95% confidence limits derived therefrom are subjected to a threshold criterion to determine the quality of the linear dependence. Regressions are performed for this in practice iteratively with different time interval lengths and time interval positions until a time interval is found in which the confidence interval for the calculated elastance value is below a preset minimum. This happens when Pₘᵤₛ(t) is sufficiently constant in the interval thus found and a considerable change in volume takes place at the same time, so that it is possible to determine the elastance without interference by the muscle activity.

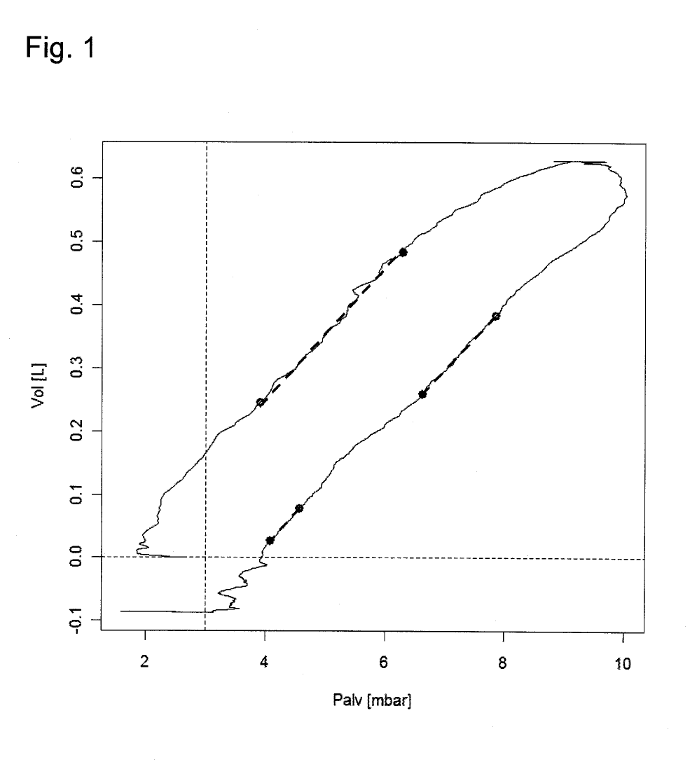

FIG. 1 shows a representation of the volume as a function of the alveolar pressure for an exemplary breath. The dependence of the alveolar pressure on the volume was analyzed with a respirator according to the present invention in many successive steps while varying time interval lengths and the positions of the time intervals by performing a linear regression of the dependence for each successive time interval and determining an indicator for the quality and subjecting it to a threshold criterion, using in this case the difference of the 95% confidence limits for the value determined for the elastance E in the linear regression, standardized for the root of the number of data values in the time interval and the value determined for the elastance E, and it was required that this value be below a preset minimum. The time intervals determined thereafter are all marked by their start point (unfilled circle) and end point (filled circle). The bold broken lines between the respective start and end points represent the regression lines, from which the elastance is calculated. The regression lines determined are essentially parallel to one another, i.e., the elastance values determined in the time interval in question have hardly any variance.

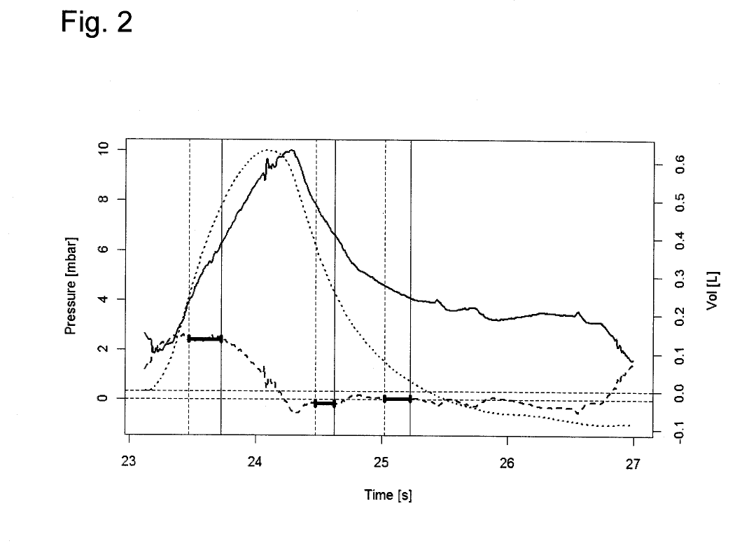

FIG. 2 shows a representation of the time dependence of the value/volume (Vol(t), dotted line) of the alveolar pressure (Pₐₗᵥ(t), solid line) and of the estimated muscle pressure (Pₘᵤₛ(t), curve in broken line) for the same exemplary breath as in FIG. 1. The time intervals determined according to the present invention are marked by their respective start points (solid vertical line) and end points (solid vertical line) as well as by bold horizontal bars. The comparison with the curve of the estimated muscle pressure Pₘᵤₛ(t) shows that the time intervals determined on the basis of the dependence of Pₐₗᵥ(t) and Vol(t) do, indeed, identify time intervals in which the muscle pressure Pₘᵤₛ(t) calculated subsequently does not essentially change.

While specific embodiments of the invention have been described in detail to illustrate the application of the principles of the invention, it will be understood that the invention may be embodied otherwise without departing from such principles.

What is claimed is:

1. A respirator comprising:
a fan for feeding breathing gas with an adjustable pressure or volume flow curve;
a recording means for recording measured values for airway pressure (P_{aw}(t) and volume flow per time (Flow(t));
a control and analyzing means to determine resistance (R) and to determine alveolar pressure (Pₐₗᵥ(t)) by subtracting a resistive pressure component (Flow(t)*R) from the measured airway pressure (P_{aw}(t)) and to plot the alveolar pressure Pₐₗᵥ(t) as a function of time, to analyze the functional dependence of alveolar pressure (Pₐₗᵥ(t)) and tidal volume (Vol(t)) for checking a functional dependence of Pₐₗᵥ(t) and Vol(t) with variable time intervals for time intervals in which an indicator for the quality of a linear functional dependence of Pₐₗᵥ(t) and Vol(t) meets a preset threshold criterion, and to determine an elastance (E) or a compliance (C) from the rise of the alveolar pressure (Pₐₗᵥ(t)) as a function of the tidal volume (Vol(t))) only in the time intervals thus determined.

2. A respirator in accordance with claim 1, wherein the control and analyzing means performs linear regressions in the time intervals with variable interval lengths as an indicator of the quality of the linear functional dependence of Pₐₗᵥ(t) and Vol(t) and to subject a function of the residues of the linear regression, the variance, to a preset threshold criterion.

3. A respirator in accordance with claim 2, wherein the control and analyzing unit determines the variance of the linear regression and therefrom the confidence limits for a preset percentage, preferably the 95% confidence limit, and subjects the confidence limits to a preset threshold criterion.

4. A respirator in accordance with claim 3, wherein the control and analyzing means forms a difference of the confidence limits as an indicator for the quality of the regression and to standardize this difference for the root of the number of data values in the time interval and for the value determined in the regression for the elastance and to require as a threshold criterion that the result be less than a preset value.

5. A respirator in accordance with claim 1, wherein the control and evaluating unit performs linear regressions in the time intervals with variable interval lengths as an indicator of the quality of the linear functional dependence of Pₐₗᵥ(t) and Vol(t) and uses the correlation coefficient of the respective regressions as an indicator of quality, wherein a minimal deviation of the correlation coefficient from 1 is allowed as a preset threshold criterion.

6. A respirator in accordance with claim 1, wherein the control and analyzing means performs tests of the hypothesis that the dependence is a linear dependence in the time intervals with variable interval lengths as an indicator of the quality of the linear functional dependence of Pₐₗᵥ(t) and Vol(t) and requires a significance level of at least 95% for testing the hypothesis as a preset threshold value.

7. A respirator in accordance with claim 1, wherein the control and analyzing means, upon determining a plurality of time intervals meet the threshold criterion within one breath cycle, sums up the values calculated for compliance or elastance in these time intervals by performing at first a freak value test and then discarding detected freak values, and summing up the remaining values into a mean or median value.

8. A respirator in accordance with claim 1, wherein the control and analyzing means sums up the values calculated for compliance or elastance in a plurality of time intervals over consecutive breaths by performing at first a freak value test and discarding detected freak values and summing up the remaining values into a mean or median value.

9. A respirator in accordance with claim 1, wherein the control and analyzing means determines resistance and elastance/compliance to set the degree of assist in proportional pressure support or proportional assist ventilation.

10. A respirator in accordance with claim 1, wherein the control and analyzing means calculates the pressure Pₘᵤₛ(t) generated by respiratory muscle activity according to the relationship Pₘᵤₛ(t)+P_{aw}(t)=R*Flow(t)+E*Vol(t)+PEEPi, where PEEPi is the intrinsic PEEP (positive end-expiratory pressure).

11. A process for automatically operating a respirator, the process comprising the steps of:
providing a fan for feeding breathing gas with an adjustable pressure or volume flow curve;
measuring values for airway pressure P_{aw}(t) and volume flow Flow(t) for the fed breathing gas;
recording the measured values for the airway pressure P_{aw}(t) and the volume flow Flow(t) in a control and analyzing unit;
determining tidal volume Vol(t) and resistance R;
determining alveolar pressure Pₐₗᵥ(t) by subtracting the resistive pressure component Flow(t)*R from the measured airway pressure P_{aw}(t) and plotting the determined alveolar pressure Pₐₗᵥ(t) as a function of time;
analyzing the functional dependence of Pₐₗᵥ(t) and Vol(t) in the control and analyzing unit including checking the functional dependence of Pₐₗᵥ(t) and Vol(t) with variable time interval lengths for time intervals in which an indicator of the quality of a linear functional dependence of Pₐₗᵥ(t) and Vol(t) is above a preset threshold;
determining elastance or compliance from the rise of the alveolar pressure Pₐₗᵥ(t) as a function of the volume Vol(t) only in the time intervals thus determined; and
controlling, with the control and analyzing unit, the fan to generate a pressure curve defined as a function of the determined elastance or compliance.

12. A respirator comprising:
a fan for feeding breathing gas with an adjustable pressure or volume flow curve;
a recording means for measuring values for the airway pressure (P_{aw}(t)) and volume flow per time (Flow(t)) and recording the measured values for the airway pressure (P_{aw}(t)) and volume flow per time (Flow(t));
a control and analyzing means for determining tidal volume Vol(t) and resistance R;
determining alveolar pressure Pₐₗᵥ(t) by subtracting the resistive pressure component Flow(t)*R from the measured airway pressure P_{aw}(t) and plotting the determined alveolar pressure Pₐₗᵥ(t) as a function of time; analyzing the functional dependence of Pₐₗᵥ(t) and Vol(t) in the control and analyzing unit including checking the functional dependence of Pₐₗᵥ(t) and Vol(t) with variable time interval lengths for time intervals in which an indicator of the quality of a linear functional dependence of Pₐₗᵥ(t) and Vol(t) is above a preset threshold; determining elastance or compliance from the rise of the alveolar pressure Pₐₗᵥ(t) as a function of the volume Vol(t) only in the time intervals thus determined; and controlling, with the control and analyzing unit, the fan to generate a pressure curve defined as a function of the determined elastance or compliance.

13. A respirator in accordance with claim 12, wherein the control and analyzing unit performs linear regressions in the time intervals with variable interval lengths as an indicator of the quality of the linear functional dependence of Pₐₗᵥ(t) and Vol(t) and to subject a function of the residues of the linear regression, the variance, to a preset threshold criterion.

14. A respirator in accordance with claim 13, wherein the control and analyzing unit determines the variance of the linear regression and therefrom the confidence limits for a preset percentage, preferably the 95% confidence limit, and subjects the confidence limits to a preset threshold criterion.

15. A respirator in accordance with claim 14, wherein the control and analyzing unit forms a difference of the confidence limits as an indicator for the quality of the regression and to standardize this difference for the root of the number of data values in the time interval and for the value determined in the regression for the elastance and to require as a threshold criterion that the result be less than a preset value.

16. A respirator in accordance with claim 12, wherein the control and evaluating unit performs linear regressions in the time intervals with variable interval lengths as an indicator of the quality of the linear functional dependence of Pₐₗᵥ(t) and Vol(t) and uses the correlation coefficient of the respective regressions as an indicator of quality, wherein a minimal deviation of the correlation coefficient from 1 is allowed as a preset threshold criterion.

17. A respirator in accordance with claim 12, wherein the control and analyzing unit performs tests of the hypothesis that the dependence is a linear dependence in the time intervals with variable interval lengths as an indicator of the quality of the linear functional dependence of Pₐₗᵥ(t) and Vol(t) and requires a significance level of at least 95% for testing the hypothesis as a preset threshold value.

18. A respirator in accordance with claim 12, wherein the control and analyzing unit, upon determining a plurality of time intervals meet the threshold criterion within one breath cycle, sums up the values calculated for compliance or elastance in these time intervals by performing at first a freak value test and then discarding detected freak values, and summing up the remaining values into a mean or median value.

19. A respirator in accordance with claim 12, wherein the control and analyzing unit determines resistance and elastance/compliance to set the degree of assist in proportional pressure support or proportional assist ventilation.

20. A respirator in accordance with claim 12, wherein the control and analyzing unit calculates the pressure Pₘᵤₛ(t) generated by respiratory muscle activity according to the relationship Pₘᵤₛ(t)+P_{aw}(t)=R*Flow(t)+E*Vol(t)+PEEPi, where PEEPi is the intrinsic PEEP (positive end-expiratory pressure).
